(19) United States
(12) Patent Application Publication
(10) Pub. No.: US 2021/0280315 A1
(43) Pub. Date: Sep. 9, 2021

(54) DATA-BASED GENERAL HEALTH CERTIFICATION FOR A PRODUCT LIFE CYCLE

(71) Applicant: Visualogyx, Inc., Aventura, FL (US)

(72) Inventor: Jorge Woldenberg, Aventura, FL (US)

(21) Appl. No.: 16/898,322

(22) Filed: Jun. 10, 2020

Related U.S. Application Data
(60) Provisional application No. 62/994,124, filed on Mar. 24, 2020.
(60) Provisional application No. 62/985,800, filed on Mar. 5, 2020.

(51) Int. Cl. G16H 50/30; G06F 17/18; G16H 40/67; G16H 50/70; G16H 80/00; H04L 29/08

(52) U.S. Cl. CPC G16H 50/30; G06F 17/18; H04L 67/306; G16H 50/70; G16H 80/00; G16H 40/67

(57) ABSTRACT

Introduced here are technologies for providing a general health certificate for each stage of a product life cycle and for the life cycle as a whole. The platform utilizes sensors at each stage of the life cycle and sensors tracking the health of participants in the life cycle. The data from the sensors are analyzed to determine the probability of viral cells and/or pathogens contacting the product. The general health certificate, depending on the probability that viral cells and/or pathogens contacted the product, will indicate a safe or unsafe result. The general health certificate may change throughout the life cycle depending on whether the health risk continues to exist. Additionally, the platform may provide recommendations to avoid the spread of any detected viruses. The platform can be implemented as a plug-in and/or solitary application. The goal is to prevent the spread of a virus.

### CROSS-REFERENCE TO RELATED APPLICATION(S)

This application claims priority to U.S. Provisional Application No. 62/985,800, titled “Generating a Verifiable and Secure Report for Tracking a Product Life Cycle” and filed on Mar. 5, 2020 and to U.S. Provisional Application No. 62/994,124, titled “Plug-in for Generating Reports During a Product Life Cycle” and filed on Mar. 24, 2020.

### TECHNICAL FIELD

The disclosed teachings related to providing a health certification for a product. More specifically, to providing health certifications based on data from each stage of the product life cycle.

### BACKGROUND

Humans have been dealing with diseases for thousands of years. We have found varying degrees of success in coping with diseases depending on the severity and ease of infection. In many cases, we now have vaccines and other effective treatments for dangerous diseases. For example, we now have vaccines and/or treatments for smallpox, measles, typhoid, cholera, polio, rubella, and several others.

However, the process from discovery of the disease to a vaccine takes several years. Additionally, millions, if not billions, of dollars are invested in the process. Most studies estimate between 12 to 20 years and an average of $500 million for a vaccine to be deployed to the general public. However, these estimates are assuming that the vaccines succeed at every stage, without multiple attempts. As such, the costs of development places a hefty burden on the general public in the form of a substantial risk to one's health in the interim.

Due to the risk to one's health, humans have adopted various methods to slow the spread of a new diseases. For example, we have mandated quarantines, face masks, banned travel, etc. In other words, depending on the severity and risk to human life, we have gone as far as to confining people to their own homes and allowing departure only for essential tasks. Although these measures have proven to be effective mitigators and are accepted with the general well-being in mind, it shows that we have continued to apply rudimentary solutions to an extremely complicated problem.

### BRIEF DESCRIPTION OF THE DRAWINGS

Embodiments of the present invention will be described and explained through the use of the accompanying drawings.

FIG. 1 illustrates an example of a display regarding the health certifications awarded to stages of a product life cycle.

FIG. 2 is a flowchart with a set of operations for analyzing health-related data and presenting recommendations which address the issues.

FIG. 3 is a flowchart with a set of operations for flagging health issues based on inputs from participants.

FIG. 4 is an example of a graphical user interface that allows the user to scan a quick response code to get health related information about a product.

FIG. 5 is an example of a graphical user interface that allows a user to self-report their health status.

FIG. 6 illustrates an example of network-based environment in which some embodiments may be utilized.

FIG. 7 illustrates a block diagram of an exemplary plug-in embodiment of the present invention.

FIG. 8 is a block diagram illustrating the components of an embodiment of a health certification module.

FIG. 9 illustrates an example of a computer system with which embodiments of the present invention may be utilized.

The drawings have not necessarily been drawn to scale. For example, the dimensions of some of the elements in the figures may be expanded or reduced to help improve the understanding of the embodiments of the present invention. Similarly, some components and/or operations may be separated into different blocks or combined into a single block for the purposes of discussion of some of the embodiments of the present invention. Moreover, while the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Disease prevention and control have taken many forms throughout human history. In many cases the methods used depend on the status of the disease. For example, in situations where the disease is infectious, governments instill quarantines, limit interactions, limit travel, and in general, attempt to prevent a pandemic. Although such preventative measures may have been effective pre-globalization, it has become increasingly difficult to prevent a disease from crossing borders. During the 21st century, a global trade market has prevented rudimentary measures from being effective.

In the 21st century, everyday items come from outside one's community, state, and even country. As such, the movement of people and goods around the world has allowed for diseases to do so as well. Although preventative measures are taken, they are expensive, time-consuming, and/or limit a customer to a small selection of products.

Further, those skilled in disease prevention are aware that one of the biggest hurdles in pandemic prevention is testing. Particularly, it is extremely difficult to differentiate between “live” virus cells and decaying viral cells. Decaying viral cells leave behind traces of viral RNA. RNA is ribonucleic acid and is present is all living cells. Thus, when a genetic material degenerates, it leaves RNA behind. A live virus cell can infect anyone that contacts the cell. RNA, conversely, is not infectious.

As such, detecting RNA cells in an area may indicate the previous presence of a virus. The commonly known methods of detecting RNA include Northern Blot Analysis (NBA), nuclease protection assays (NPA), in situ hybridization, and reverse transcription-polymerase chain reaction (RT-PCR). NBA provides information about the endogenous transcript size and is capable of being done with various probes. NPA is ideal for multi-target analysis and mapping studies. In situ hybridization can localize gene expression within tissues or cells. RT-PCR can detect RNA even in the smallest amount in any gene. One of skill in the art will recognize that there are other applicable methods as well.

Tests similar to NBA, NPA, RT-SCR require scientists to swab various surfaces to collect particles and grow the virus in a lab, inside host cells. Such activity requires facilities with extensive capabilities, the least of which is strenuous biosafety standards. For example, a lab that grows and tests virus needs to have full -body hazard suits, clean rooms, detoxification mechanisms, contamination procedure, and much more. Moreover, RNA tests take several days to process and can be expensive. As such, a data-based approach will help approximate the existence of risk, while we wait for tests for the existence of live viral cells to process.

Introduced here are systems and methods for providing a data-based health certification for a product's life cycle stages. Generally, the disclosed embodiments are directed to a health certification platform (also referred to as “the platform”) and method for analyzing data from the product life cycle. The data is related to health risks present in any location and/or on a person that contacts the product. For instance, the data can be indicative of the existence of pathogens related to the common cold, flu, meningitis, herpes, chicken pox, measles, norovirus, rotavirus, hepatis, yellow fever, dengue fever, HIV, AIDS, or another virus. Additionally, the data can be related to outbreaks within the area, health histories of those that contact the product, presence of viral cells, and other such data. These data points can be organized based on the stage of the life cycle, such as manufacturing, transportation, testing, or designing. As a result, each stage can get its own health certification. The certification alerts a buyer that the product, for example, potentially contacted a health risk.

The health certification can be implemented as a health certification platform (i.e., a smart application or plug-in), which is connected to a network. The participant in the life cycle can interface with the health certification platform via an interface. For example, the health certification platform can be incorporated with the participant's cell phone and the interface can be cell phone screen. Accordingly, the participant can then examine content generated by the platform.

In some embodiments, the health certification platform can be configured to analyze the information generated by sensors at each stage of the life cycle and on wearable devices worn by participants and display the data for the participant to analyze. The sensors may include cameras, heart rate sensors, temperature sensors, motion sensors, biosensors, audio sensors, radiation sensors, air pollution sensors, optical sensors, potentiometric sensors, impediametric sensors, amperometric sensors, piezoelectric sensors, magnetic sensors, thermometric sensors, protein-based sensors, fluorescent sensors, luminescent sensors, interferometric sensors and/or other sensors. The sensors can include a network interface for communicating wirelessly (i.e., Wi-Fi) or with communication protocols (i.e., Bluetooth or NFC) to a server or other devices.

In some embodiments, air pollution sensors can be used in conjunction with other data to detect viruses. Air pollutant sensors, generally, focus on detecting ozone, particulate matter, carbon monoxide, sulfur dioxide, radon, and/or nitrous oxide. Moreover, virus-laden droplets from infected people's coughs and sneezes fall to the ground within a meter or two. However, smaller droplets, those that are less than 5 microns in diameter, can remain in the air for hours. These smaller droplets can then be carried to another location via air pollutants. For example, dense air pollution (i.e., carbon monoxide) in conjunction with other risk indicators (i.e., a microphone hearing a sneeze), can indicate the presence of a virus.

In some embodiments, multiple sensors can be placed at the location of every stage. Generally, sensors can be placed at the optimal position to monitor the product and the environment near the product. Placement of the sensors can vary based on, for example, whether the location is indoors, outdoors, moving, temperature controlled, separated into multiple sub-stages and/or other similar factors. The placement can also depend on the type of product being tracked (i.e., perishable or non-perishable) or the sub-stages of a larger stage. For example, a packaging facility can include multiple sub-stages such as delivery, storage, placing into boxes, tagging boxes with shipping labels, transporting to a shipping facility, and/or other sub-stages. Moreover, each sub-stage may have different equipment and people involved.

For instance, the platform may be tracking the packaging stage of oranges. The packaging stage can include taking delivery of the oranges, placing a certain amount (i.e., based on weight) into each container, sealing the container, and placing the container into a shipping vehicle. The placement of the sensors can vary at each of these sub-stages. During the delivery stage, a video camera can be placed at door of the receiving dock of the facility to monitor the employees having contact with the oranges. While placing oranges inside containers, the insides of the containers can include temperature sensors and air pollution sensors.

Similarly, a stage and its sub-stages may require different sensors based on the situation. For instance, the delivery sub-stage requires different sensors than the packaging stage. The delivery sub-stage may need to monitor the health of the delivery person, while the packaging sub-stage needs to monitor the environment in the facility and inside the container. As such, a homogeneity of sensors may not be ideal for the entire product life cycle.

In some embodiments, biosensors can be placed in various stages, such as in the stages that expose the product to the environment. For example, a set of biosensors in a manufacturing facility may have information which indicates that live SARS-CoV-2 cells or viral cells were previously or currently are present in the facility. in the nearby environment. The health certification platform may then present the information of the potential presence of SARS-CoV-2 cells to a participant in the form of an unsafe certification.

A biosensor is a device used to detect a chemical substance, which combines a biological component with a physiochemical detector. A biological component is one that interacts with, binds with, or recognizes the target substances. For example, a biological component could be tissue, organelle, cell receptors, enzymes, antibodies, etc. The detector element can be optical, piezoelectric, electrochemical, etc., which is triggered when the biological components is altered by contacting the target substance. One example of a biosensor is a BioFET sensor which can be used for detection of DNA hybridization, biomarker detection, antibody detection, etc.

In some embodiments, several sensors can be used in conjunctions. For example, an optical sensors can be paired with optical sensors, potentiometric sensors, impediametric sensors, amperometric sensors, piezoelectric sensors, magnetic sensors, and thermometric sensors. Optical sensors, generally, are based on the detection of the pathogen through the change in color or production of color during a reaction between an analyte and the target. Potentiometric sensors measure potential differences when a specific current value is applied to an electrochemical cell via a potentiostat. An impediametric sensor helps to analyze the complementary binding of DNA by measuring resistance produces due to binding of biological elements on a surface of an electrode with a target molecule. Amperometric sensors generate a current-based response when specific potential is applied to a potentiostat. Piezoelectric sensors detect a change in voltage when mechanical stress or oscillation is applied to a surface of an electrode with piezoelectric material. A magnetic sensor is made up of paramagnetic or super-paramagnetic particles, or crystals which help detect biological interactions by determining the changes in magnetic properties. A thermometric sensor measures changes in temperature caused by a biochemical reaction.

In some embodiments, an optical sensor and thermal sensor can be configured to detect COVID-19, and its particles, SARS-CoV-2. The combination of sensors can be placed, for example, on the packaging, within facilities (i.e., manufacturing, packaging, etc.), and/or other areas near the product. The combination of sensors can help detect COVID-19 because COVID-19 is an RNA virus, which means its genome does not consist of a DNA double strand as in other living organisms. Instead, it has only one RNA strand. To detect such organisms, those skilled in the art use so-called gold nanoislands placed on a glass substrate with DNA receptors grafted onto the gold nanoislands. The refractive index of the gold nanoislands change when in contact with RNA strands. Thus, an optical sensor can detect the change in the refractive index, which could indicate the presence of COVID-19. Additionally, a thermal sensor can concurrently detect temperature changes caused by denaturation and hybridization of RNA strands to more accurately assess the presence of COVID-19.

For example, a shipping container can have a glass surface. Gold nanoislands can be placed on the glass via an adhesive. An optical sensor can be placed at checkpoints along the shipping route. The checkpoints can be when the container moves to another facility, changes transportation mediums, or at other suitable times. For instance, a conveyor belt can carry the container from a loading dock to a temporary storage facility (i.e., shipping truck to a UPS facility). Optical sensors can be placed along the conveyor belt such that the glass surface is in the viewing angle of the optical sensors. Thus, the optical sensors can detect the RNA strands and alert an employee to, for example, remove the package from circulation.

In some embodiments, conductive polymers are used to detect changes in electricity caused by virus pathogens. For example, a polymer film (i.e., 3,4-ethylenedioxythiophene) can be placed between two electrodes. The polymer film acts as a attractive and/or bonding element to virus pathogens. Thus, when virus pathogens are in the environment near the polymer film, they adhere to the polymer film. Many virus pathogens such as, H1N1, carry a small positive charge on its exterior shell. This positive charge causes a small change in voltage, which the electrodes can detect. This system allows for detection of miniscule viral pathogens and enable point-of-care detection.

In some embodiments, sensors similar to breathalyzers can be used to detect whether the environment near a product or participants in the life cycle pose a threat. For example, a breathalyzer-like device can incorporate biomarkers present in a person's breath which afflicted with a particular disease. For instance, asthma sufferers have an increased concentration of nitric oxide in their breath and acetone is a known biomarker for diabetes. Moreover, it is known that nitric oxide and ammonia concentrations are higher in people inflicted with the flu. Thus, a breathalyzer, can use, for example, the polymer-based sensor mentioned herein, to detect viral pathogens in a person's breath.

The health certification platform can be accessible via web browser, desktop application, mobile application, and/or over-the-top application. As such, the interface can be personal computer, tablet computer, personal digital assistance (PDA), mobile phone, game console, music player, wearable electronic device, network-connected electronic device, virtual/augmented reality system, or some other electronic device.

The following paragraphs describe embodiments from a user-side portion of the health certification platform (also referred to as “the platform”), which is implemented as a smart application and/or plug-in. For example, each user (also referred to as buyer, end-user, or participant) can download the health certification platform on their respective network-connected devices. In some embodiments, the platform can be implemented as a plug-in which can be integrated with other applications.

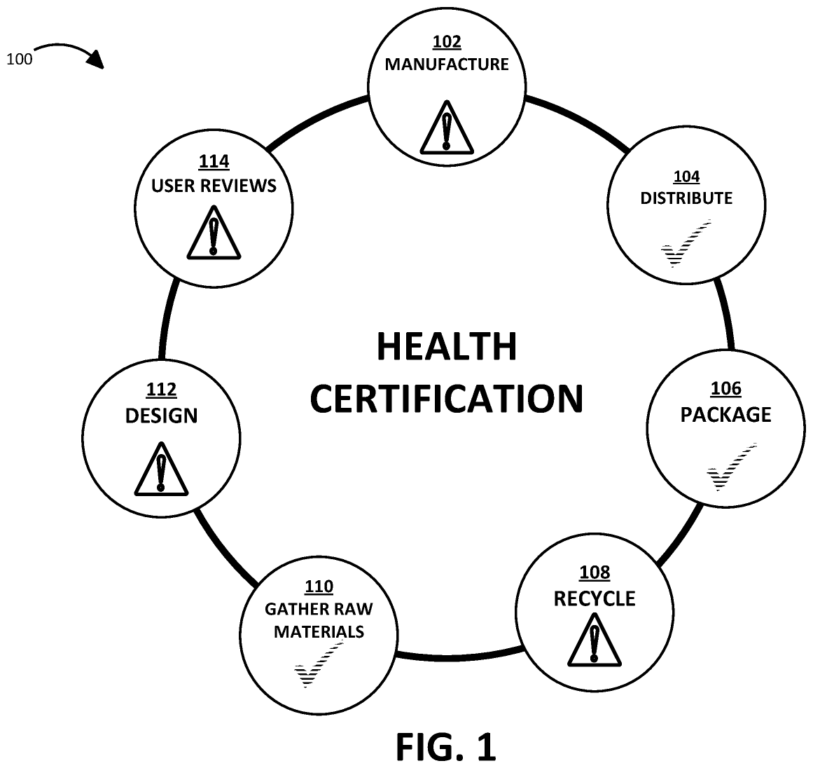

FIG. 1 illustrates an example of a display regarding the health certifications associated with stages of a product life cycle. The stages include manufacture 102, distribute 104, package 106, recycle 108, gather raw materials 110, design 112, and user reviews 114. Here, distribute 104, package 106, and gather raw materials 110 received a check mark indicating that the stages do not pose a health risk to the user. Conversely, manufacture 102, recycle 108, design 112, and user reviews 114 include a warning sign, indicating a potential health risk (i.e., potential contact with SARS-CoV-2 particles). A person of skill in the art would acknowledge that there are numerous other stages and/or phases in a product life cycle. As such, the description herein is meant to be applicable to every stage and/or phase of the product life cycle.

A health certification can be derived from multiple data points. For example, the platform can collect self-reported data, reports from government agencies (i.e., Federal Drug Administration), reviews from previous users of the product, and/or medical reports of participants in the stages. These data points can be collected to determine whether the risk continues to exist. For example, in the distribution phase 104, an employee may have taken a sick day during the time that the product was in the vicinity of the employee and subsequently, tested positive for COVID-19. In some cases, the platform may gather the data indicating that an employee took a sick day and request further information regarding the employee. However, the platform may also determine that the contact does not pose a threat to an end-user (i.e., buyer and/or participant). The determination that the end-user is not in danger can be based on, for example, the materials of the product, the number of days that have passed since the contact, the weather conditions, and other relevant conditions. Conversely, in manufacture 102, a similar event could have occurred within the last two hours. Thus, it is worth alerting an end-user to the potential risk.

In yet another example, data from user reviews can be analyzed as a factor in the health certification. For example, data from user reviews on Yelp, Amazon, Google, Twitter, Facebook, and/or other similar websites can be parsed for information. For instance, a review for a product on Amazon may indicate that a product label says the product was manufactured in an epicenter of a viral disease. The review can be factored into providing the health certification.

In some embodiments, the analysis may include monitoring sub-stages of the stages shown in FIG. 1. For example, package 106 can include multiple sub-stages based on multiple layers of packaging, different materials, packaging portions of the product, and/or other such sub-stages. For instance, a dining table package can include multiple pieces, each individually packaged, and multiple layers over a certain piece (i.e., a glass table top). As such, each layer of packaging and piece of the entire product can be monitored. The monitoring can begin prior to applying the layer of packaging to the product. For example, sensors (i.e., a camera) can be placed near the spool of bubble wrap before it is applied to a piece of the dining table. Additionally or alternatively, the sensors can monitor the layer after it is applied to the piece and prior to another layer being applied. In some embodiments, each layer of packaging and/or each piece of the product can receive its own health certificate.

In some embodiments, the health certificate may provide different certifications based on the level of risk. For example, in the case above where an employee took a sick day and subsequently, tested positive for COVID-19, the system may determine that it is a not a high risk (i.e., due to 15 days passing since contact). As such, the certification may include these details in a materials disclosure sheet or on various displays of the platform. The details can include, for example, the approximate survival time of SARS-CoV-2 cells on the material, the weather conditions that the product travelled through, and other relevant data.

In some embodiments, the platform may have predetermined values for each type of risk. The sum of these values must go beyond a predetermined threshold before the platform flags the stage as a health risk. For example, the platform may value each risk on a scale from one to ten, ten being the highest health risk. The sum of all the detected health risks may have to surpass one hundred before an alert is raised. The score of a health risk may depend on the severity of the risk, the credibility of detected risk, proximity of the risk to the product, and other factors. For example, a failing health score from a government agency (i.e., Federal Drug Administration), can receive a ten because it is a high risk from a credible source. Conversely, a review from a previous user which criticizes the cleanliness of the packaging may not be as highly valued.

In some embodiments, the health certification is a dynamic one. A dynamic health certificate allows for a health certification to change if a health risk is mitigated or newly raised. For example, during the distribution stage, the platform may not have detected any health risks; thereby, it may grant the stage a passing health certification. However, at a later time, the platform may learn that an employee of the distribution company became sick. The platform may then adjust the certification accordingly.

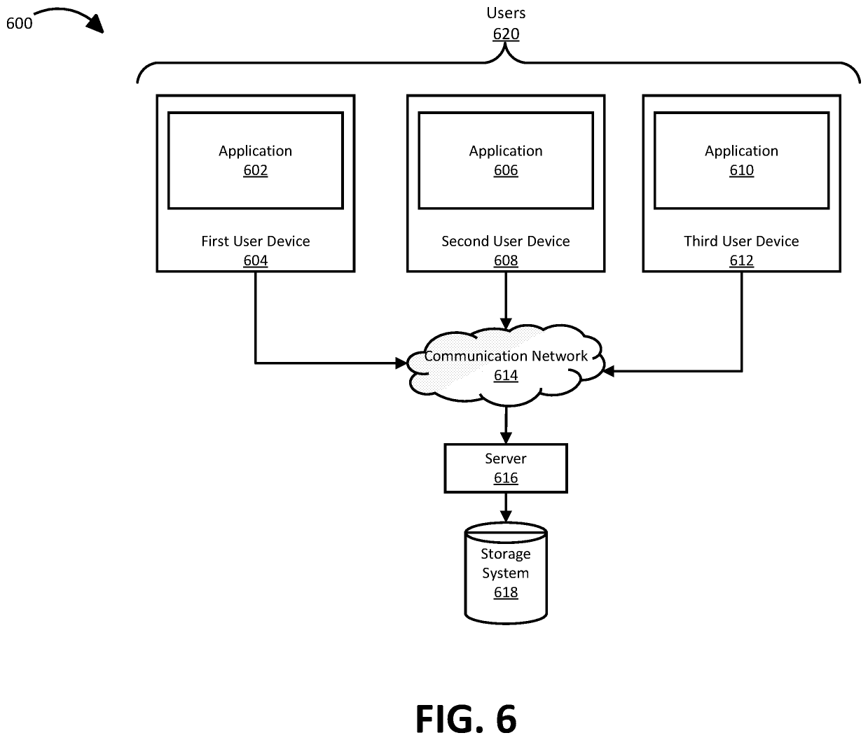

In some embodiments, the health certificate is a virtual certificate provided on the platform. As such, when a certification needs updating due to new information, the platform may update the virtual certificate and alert the user on their smart device. For example, when the platform learns that a manufacturing person tested positive for COVID-19 four days after the product left the manufacturing facility, the platform may update the virtual certification accordingly. Further, in some embodiments, the certification may be updated based on, for example, days since the potential exposure to a virus, mitigation techniques used by participants (i.e., cleaning), and other new information. The updated certifications may be reflected on the platform based on continuous analysis of the data. The results of the analysis (i.e., a new certification) can be pushed to a user's smart device. For instance, as further described in conjunction with FIG. 6, a processor can analyze the data and subsequently, via a communication network and server, can push the updated certificates to the smart application on each user's smart device.

In some embodiments, there are levels of health certifications such as no risk, potential risk, avoid, passing, failing, and/or other levels. Additionally, the platform can provide a health certification based on the days since a health risk occurred and/or based on the virus that caused the risk. For example, a participant in the life cycle may have had chicken pox and had contact with the product in the last two days. The health certification can notify the user that the risk was caused by chicken pox, two days ago. Thus, the user can then determine on their own if they should avoid the product or not.

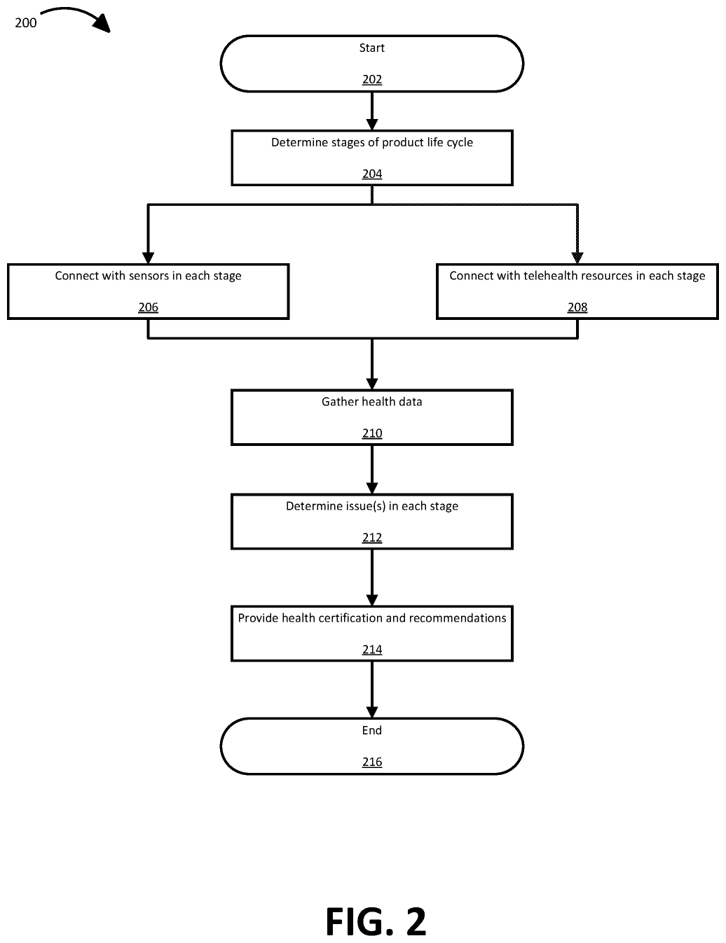

FIG. 2 is a flowchart with a set of operations for analyzing health-related data and presenting recommendations to address the issues. Recommendations flowchart 200 includes start 202, determine stages of product life cycle 204, connect with sensors in each stage 206, connect with telehealth resources in each stage 208, gather health data 210, determine issue(s) in each stage 212, provide health certification and recommendations 214, and end 216.

At the block 204, the platform determines the stages of a product life cycle. The stages can be, for example, pre-determined, input by a user, and/or derived from the collected data. The stages can include, for instance, collecting raw materials, designing, manufacturing, recycling, and/or distribution. By separating the life cycle into stages, the platform can designate data to each stage, and thereby, determine the health certification for each stage.

In some embodiments, the stages are determined by location and/or time. For example, while a product is in a facility in location A, it may be in a first stage. Subsequently, when the product is moved to a facility in location B, it may be in a second stage. Additionally or alternatively, the stages can be determined by time. For example, a user can input the timeline of the product lifecycle. For instance, manufacturing for three days, testing for two days, packing for one day, and shipping for two days. Thus, when the platform begins to monitor a product, it may follow a pre-determined timeline for each stage.

At block 206 and 208, the platform may gather information from sensors and telehealth resources. For example, a manufacturing facility for water bottles may include multiple sensors for temperature, biohazards, radiation, and the like. Thus, if a biosensor, such as an arsenic sensor, detects arsenic, the sensor can transmit this data to an external device (i.e., server or processor) which can relay similar information to the platform.

For instance, a microphone at a packaging facility may hear an employee sneeze. A particulate matter sensor may detect particulate matter (i.e., Styrofoam) in the air, as a byproduct of packaging activity. Thus, the platform can combine the data from the microphone and particulate matter sensor, to detect a potential threat that a virus may travel throughout the packaging facility.

Combining data, as described above, can be done in multiple ways. Generally, the platform can combine data based on, for example, the time that data was collected, the location from which the data was collected, and/or other factors. The platform can apply methods such as Record Linkage algorithms and/or other techniques. Record linkage methods associate data points that are thought to belong to the same entity. Almost any variable that is present in two or more data points can be used as a linkage variable. Thus, for example the location from where the data was collected can link data points from different sensors.

In some embodiments, data can be compartmentalized based on metadata. Metadata is generally defined as data that provides information about other data. Metadata can include data such as descriptive metadata, structural metadata, administrative metadata, reference metadata, and/or statistical metadata. In particular, metadata can include the means of creation of the data, purpose of the data, time and date of creation, creator or author of the data, location of the computer network where the data was created, file size, data quality, and much more. Thus, for example, data can be grouped for analysis based on being collected by an optical sensor and temperature sensor that were both triggered by motion sensor. Through such grouping or combining techniques, the platform can better detect a health threat.

Similarly, at block 208, the platform can gather information from telehealth resources. For example, from a medical provider, telehealth smart applications, medical insurance provider, or the like. A participant can grant access to her medical records. In some embodiments, the access to telehealth sources can be limited. For example, a participant may only provide access for a particular time period, to only certain records, and/or merely to activity such as logging on and off.

For instance, a participant may use telehealth applications such as MDLIVE, Lemonaid, LiveHealth, Express Care, PlushCare, Doctor-On-Demand, or the like. The platform can be granted access to one or more of these applications to analyze the participant's health status. In some embodiments, a user can selectively grant access to telehealth applications. For example, a user can self-report data from telehealth application to the platform. In other words, a user can input (i.e., self-report) their own data collected from telehealth applications. In some embodiments, the platform can communicatively link to telehealth applications. For example, a telehealth application can be embedded into the platform. Embedding a telehealth application into the platform can be done by, for example, applying Object Linking and Embedding principles and/or using a telehealth application as an add-on software. The user can subsequently access the telehealth applications through the normal log-in procedure.

At block 210, the platform gathers the relevant details from the sensors and telehealth resources. In some embodiments, there are other sources such as self-reporting. Subsequently, at block 212, the platform determines whether the stage contains any health risks. In some embodiments, the platform continuously evaluates potential health risks throughout the product life cycle and updates the health certification of each stage accordingly. In other words, recommendation flow chart 200 becomes an iterative process for each stage. As such, the platform gathers information periodically and updates the health certification accordingly. For instance, a product may be in the manufacturing stage for three days. During those three days, the platform may pull data twice a day and continuously update the health certification. Simultaneously, the platform may also pull data for the next stage (i.e., packaging) in preparation for the product to go into that stage. Moreover, the health risk of a particular stage can continue to be evaluated even after the product moves to another stage.

In some embodiments, the determination of health risk of a stage is static once the product leaves the stage. However, while the product is in the particular stage, there can be periodic assessment of health risk. At block 214, the platform provides a health certification and recommendations to address the health risks, if any. Generally, a health certificate provides insight into potential health risks of coming into contact with the product.

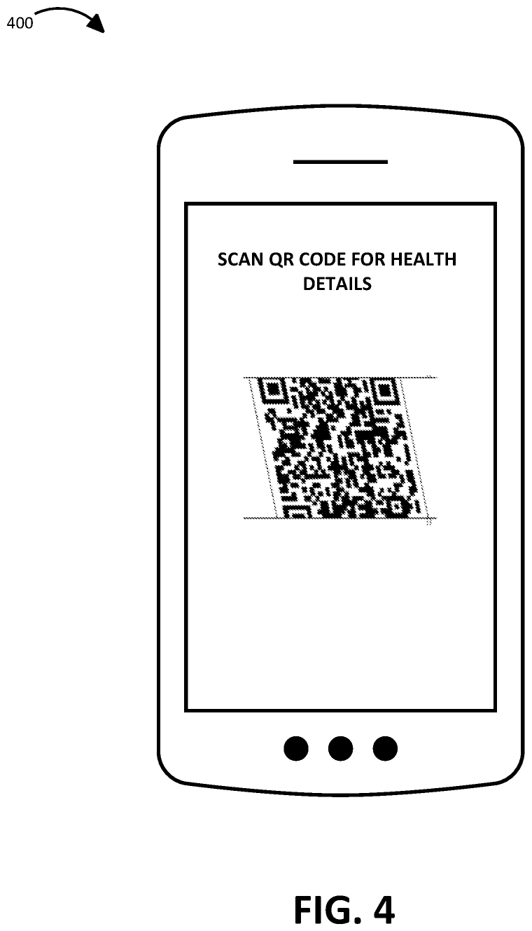

The health certification can come in various forms such as simple indicator (i.e., green check mark) printed on the product or packaging, or a detailed description provided on a website, web application, smart application, or the like. In some embodiments, the product may provide a means to access further information (i.e., QR code as further described in conjunction with FIG. 4). In some embodiments, the recommendations can mitigate the risk to a participant. For example, a recommendation could be to stay six feet away, wear gloves, wear a mask, clean the product with disinfectant, wait a particular number of days, and/or return the product. After which, the process ends at block 216.

In some embodiments, the recommendations can be in the form on a report. The report can be appended to various sources that allow participants in the life cycle and the end-user to access the report. For example, the report can be incorporated into a web-based application that can be access via any network-connected device (i.e., laptop, tablet, cellular phone, etc.). In another example, the report can be appended to a blockchain ledger. For instance, the packaging on the product can include a QR code which allows a person to access the report. The QR can give access to a web-based application on a user's phone, for example.

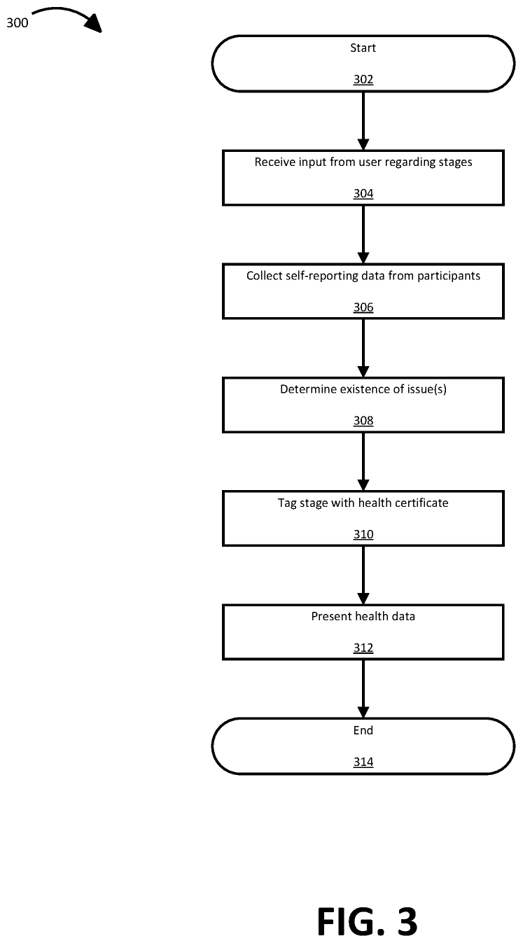

FIG. 3 is a flowchart with a set of operations for flagging health issues based on inputs from participants. User input flowchart 300 includes start 302, receive input from user regarding stages 304, collect self-reporting data from participants 306, determine existence of issue(s) 308, tag stages with health certificates 310, present health data 312, and end 314.

At block 304, a participant inputs the stages of a product life cycle. For example, a manufacturer of a cell phone may input different stages than a manufacturer of a water bottle. At block 306, the platform gathers data from self-reporting. A participant may be required to periodically self-report their health status. For example, the employee who delivers a package may have to self-report before and after delivering the package. Moreover, the participants may also provide reports of other threats, such as a sick co-worker.

At block 308, the platform can determine whether health risks (i.e., a participant diagnosed with COVID-19) are present, based on the collected data. Based determining whether a health risk exists, each stage can be tagged with a health certification at block 310. Moreover, a detailed analysis can be provided at block 312. The analysis can include granular details regarding, for example, location of the threat, time the threat occurred, and/or frequency of occurrence. Additionally, the analysis can include details about the threat such as the medical name of a detected virus, known treatments, severity of the threat, and/or common symptoms of infection.

FIG. 4 is an example of a graphical user interface that allows the user to scan a quick response (QR) code to get health related information about a product. In some embodiments, a QR code can be printed onto a product or other items provided with the product. A participant can scan the code using their network-connected smart device, to access more details. Additionally and alternatively, a hyperlink (URL) or near field communication (NFC) tag may also be placed on the product or other items provided with the product.

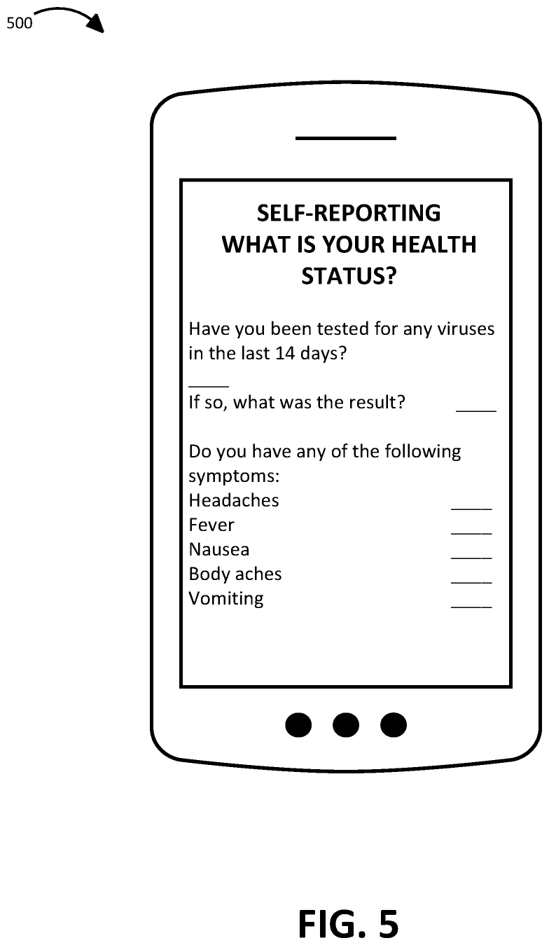

FIG. 5 is an example of a graphical user interface that allows a user to self-report their health status. In some embodiments, the self-reporting feature includes a questionnaire with various health related questions. In some embodiments, the self-reporting feature can retrieve data (i.e., heart rate) from wearable devices. For example, an employee in a manufacturing facility may be wearing a heart rate monitor on her wrist. The heart rate monitor may be connected via Bluetooth to her network-connected mobile device. As such, a health certification application on the mobile device may collect the heart rate data.

FIG. 6 illustrates an example of network-based environment in which some embodiments may be utilized. The environment 600 includes a server 616, which implements at least a portion of the health certification platform and facilitates exchanging of data (i.e., location, pictures, sensor measurements) between users 620 of the health certification platform. The users 620 can utilize the health certification platform as a smart application, for use on network-connected devices such as first user device 604, second user device 608, and third user device 612. Server 616 and/or storage system 618 can store an executable file for generating the application. The executable file can also be stored at a different location. Users 620 can subsequently download the application on their devices (i.e., 604, 608, and 612). The user device 604, 608, and 612 can be any network-connected computing device capable of accessing the server 616 over a communication network 614 and capable of sending necessary report data, such as a laptop, smartphone, tablet, etc.

In some embodiments, the health certification platform can function as a plug-in or add-on onto other security-related platforms. For example, the platform (i.e., plug-in) detailed in this application can augment shipping and logistics products such as and/or similar to Clear, Clearview AI, Malomo, Logitude World, ShipStation, and others. The platform can also be integrated with delivery services such as and/or similar to UPS, FedEx, DHL, DoorDash, Uber Eats, Postmates, GrubHub, etc.

In some embodiments, the application can be incorporated into other stages of a product life cycle (i.e., manufacturing, shipping).

For example, first user of first user device 604 can download application 602 onto first user device 604 (second and thirds users can act similarly). By doing so, users can track, report, and communicate with each other as required and/or be prompted to provide sufficient information in accordance with reporting guidelines. For example, a first user can be the buyer, a second user can be the manufacturer, and a third user can be the transporter. Each user can provide details as required by the health certification platform or by any other user.

Non-compliance with the requirements of the application (and other users 620), can result in several repercussions. For example, on the back end, server 616 may store on storage system 618 logs of activity of the users 620. The logs may include data of the completion of tasks, check points, or the like. The data from the logs can be made available to a manufacturer when selecting other supply chain participants to work with. For example, a manufacturer may be presented with a supplier that complies with the requirements of the health certification platform and other users 98% of the time. The manufacturer may also be presented with another supplier that only complies 40% of the time. Thus, the manufacturer is more inclined to select the first supplier.

In some embodiments, the platform may require users to accept a health certification contract prior to using the platform. For example, the contract may require certain tasks, checkpoints, reporting guidelines, it may delineate the repercussions of non-compliance, and other relevant information. In some embodiments, the contract may vary based on the goals of the users. Other functionalities of the application include sending and/or receiving pictures, videos, bills of lading, purchase order forms, user profiles, location, audio, and other relevant data. For example, a truck driver crossing an international border can present the border crossing agent with the data from the platform. The platform may show the agent details of the health risks. Thus, allowing a border crossing agent to limit the transfer of a virus across borders.

In some embodiments, the application can use various forms of verification to ensure that the data and/or information provided is authentic and reliable. The application can use a multi-category verification process. The categories can include single source verification, dual source verification, multi-source verification, and/or blockchain verification. For example, multi-source verification can use metadata.

In some embodiments, different participants in the life cycle may have different levels of access and requirements in the platform. For example, a buyer may only be able to view the health certifications and causes of the health certification. A participant, such as the delivery person, may only be able to see the health certification from previous stages and be required to self-report any health risks. In some embodiments, each participant may make a profile on the platform. The profile can be associated with one or more products which the participant may contact. Thus, the data regarding a particular participant may be automatically associated with every product on their profile.

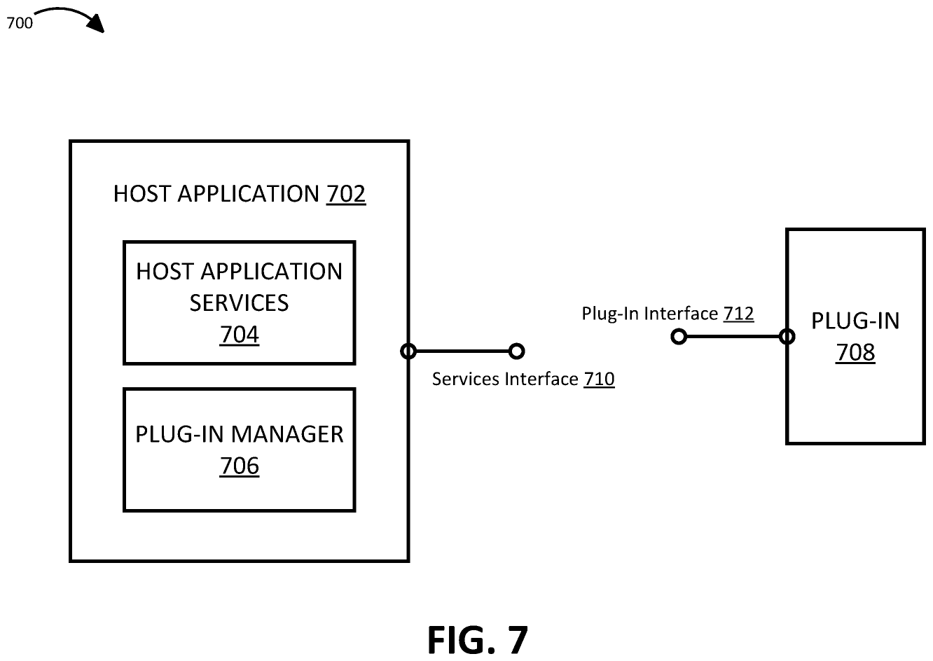

FIG. 7 illustrates a block diagram of an exemplary plug-in embodiment of the present invention. Integration diagram 200 includes host application 702, host application services 704, plug-in manager 706, plug-in 708, services interface 710, and plug-interface 712. Host application 702 can be any application and/or software related to health, tracking, reporting, shipping, logistics, delivery, security, and/or the like. A plug-in is software that extends or changes the behavior or interface of an existing, compiled application and/or adds a specific feature to an existing computer program. Generally, plug-ins are used for extending an application's functionality, changing the user interface of an application, extracting data from an application, reducing the size of an application, and/or enabling third parties to create abilities which extend an application.

Host application 702 can include host application services 704 such as facial recognition, geolocation, health report data, self-reporting data, and other services required to utilize an application or software for health purposes. Plug-in manager 706 can help manage the interaction between the plug-in and host application. In some embodiments, the plug-in manager 706 can includes the rules for what data and how much data is to be shared and/or visible to both the host application 702 and the plug-in 708.

Plug-in can connect to host application 702 via services interface 710 and plug-in interface 712. In some embodiments, connecting to a plug-in can be accomplished by an application programming interface (“API”). An API is a computer interface exposed by a particular software program, library, operating system or internet service, to allow third parties to use the functionality of that software application. For example, an API can be developed to integrate plug-in 208 with a host application 702 (i.e., Clearview).

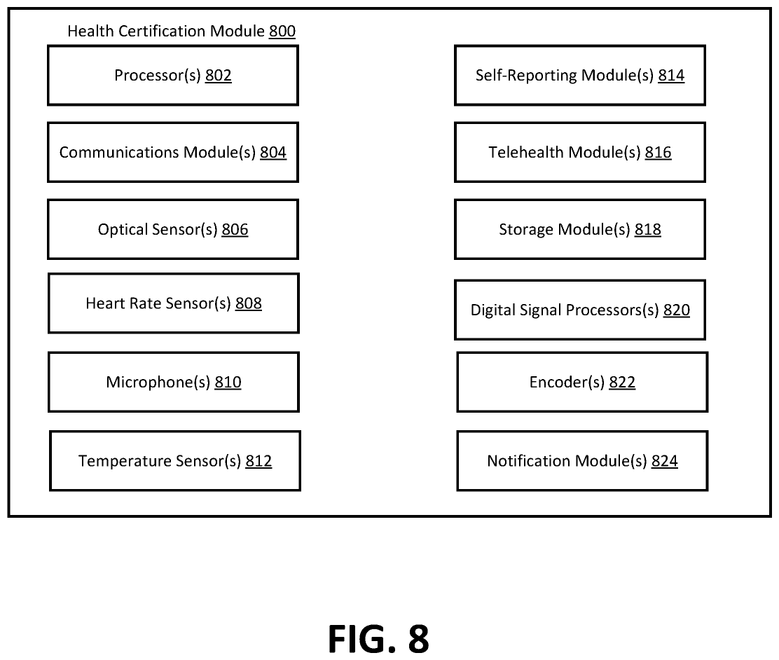

FIG. 8 is a block diagram illustrating the components of an embodiment of a health certification module. The health certification module 800 includes one or more processors 802, communication modules 804, optical sensors 806, heart rate sensors 808, microphones 801, telehealth modules 812, self-reporting modules 814, temperature sensors 816, storage modules 818, digital signal processor(s) 820, encoders 822, and notification modules 824.

In some embodiments, health certification module 800 can collect data from the various other modules to form a probability that a stage, sub-stage, packaging layer, etc. is a health risk. Generally, probability is a numerical representation of the likelihood that an event will occur or that a proposition is true. In relation to COVID-19 detection as described herein, each anomaly in the data can be assigned a value, once the sum of the values is higher than a pre-determined threshold, then the platform can issue an unsafe health certificate. For example, during a packaging phase, the platform may be programmed to allow ten data anomalies to occur prior to issuing an unsafe health certificate. Thus, health certification module 800 can analyze the data and if it detects more than ten data anomalies, then issue an unsafe health certification.

The processor(s) 802 can execute instructions stored in the storage module(s) 818. The communication modules 804 can manage communication between various components of the health certification module 800. The communication modules 804 can also manage communication between health certification module 800 and other devices. For example, communication modules 804 may facilitate communication with a mobile phone, tablet computer, wireless accept point (WAP, etc.). As another example, the communication module 604 may facilitate communication between participants in the product life cycle.

The optical sensors 806 can be configured to generate optical data from each stage of the product life cycle. Optical sensors 806 can be, for example, charged-coupled devices (CCDs), complementary metal-oxide-semiconductors (CMOSs), infrared detectors, etc. In some embodiments, the optical sensor 806 can be configured to generate a video or image of a product during a stage. For example, an optical sensor may generate video of a product during a stage to monitor who comes into contact with the product. The data may then be sent to processor(s) 802 to determine who contacted the product and retrieve their health information.

In some embodiments, an optical sensor (i.e., a camera) can capture a picture of someone that comes into contact with the product. The optical sensor can take a picture based on, for example, a motion sensor being triggered. The health certification module 800 can then analyze the image to detect health risk by analyzing, for example, the person's skin, expression, age, and other relevant information. To perform such an analysis, health certification 800 may have been training in an iterative manner, with simulated images. The simulated images may include images of people having various skin tones, facial features, illnesses, etc. Additionally, the simulated images can be taken in various lighting settings such as outdoor, indoor, daytime, night time, etc. During training, large amounts of reference images can be compared with altered images (i.e., images of sick people). By comparing the reference images to the altered images, the health certification platform 800 can be trained to detect illnesses.

In some embodiments, the health certification module 800 can use various models or algorithms to evaluate whether a person's picture shows sign of illness. A method or algorithm can be a collection of logic, data, and the like which allow for evaluation of an image. Examples of such methods include Mean-Squared-Error (MSE). MSE measures the average squared difference between actual and ideal pixel values. Another method is peak Signal-to-Noise Ratio (pSNR) calculation which indicates the ratio of pixel intensity to the distortion.

Additionally, a method can be blind or reference-less. In these methods, a high-quality image or raw data is used as a reference to compare against a taken picture. These methods are pixel-based, parametric or bitstream based, or a hybrid thereof. An example of a reference-less model is Blind/Reference-less Image Spatial Quality Evaluator (BRISQUE). The BRISQUE model is trained by using a database of images of people with known distortions. The model uses image statistics to quantify the distortions. Another reference-less model is the Natural Image Quality Evaluator (NIQE) model. NIQE is trained based on a database of flawless images. Thus, a NIQE can identify distortions by analyzing the flawless images.

Furthermore, similar training methods and algorithms can be used to detect illnesses based on data received from other sensors or devices. For example, the health certification module 800 can apply anomaly detection techniques such as k-nearest neighbor, local outlier, isolation forests, Bayesian Networks, Cluster Analysis, and other similar techniques. For example, a microphone in a manufacturing facility may hear constant noise throughout the day. The noises can include machinery, employees, outside noise, etc. However, by using the training mechanisms mentioned herein, the health certification module 800 can detect anomalies that signify an illness, such as multiple coughs.

In some embodiments, the health certification module 800 can parse the data through a pipeline of machine learning or deep learning models. For example, health certification module 800 can use any two of: a model that uses a frequency of occurrence of a term in a document, a model that derives topic features, a clustering-based model, an iterative clustering-based model, or a model that uses tree-structures for attribute classification. Examples of a model that uses frequency of the occurrence can be a term frequency inverse document frequency (TF-IDF) algorithm, the model that derives topic features comprises a Latent Dirichlet Algorithm (LDA) model, the clustering based model comprises a KMeans algorithm, the iterative clustering based model comprises a KMeans Anomaly Detector System, and the model that uses tree-structures for attribute classification comprises a Random Forest anomaly detector model. The resulting anomalies detected from the pipeline can indicative of an illness.

For example, health certification module 800 can collect data from a thermal sensor inside a shipping container. The data can be passed through a pipeline using LDA and TF-IDF. The output from the pipeline can identify temperature anomalies. Additionally, the metadata from the thermal sensor can indicate the time of the temperature anomalies. By using the metadata, the health certification module 800 can then look at data from other sensors at those times, to verify the existence of a health risk. For instance, the camera might show that an employee of the shipping company entered the shipping container at that time. Additionally, an optical sensor may detect a change in the refractive index of gold nanoislands placed inside the shipping container (described above). Subsequently, the health certification module 800 can flag the shipping stage and the product as a health risk.

Heart rate sensor(s) 808 may monitor the heart rate of participants in the product life cycle. For example, a shipping company may mandate that its employees wear heart rate sensors. Subsequently, the company may provide heart rate data of its employees to health certification module 800. This may allow for increased transparency regarding the potential health risks. Microphone(s) 810 may be incorporated into the locations where the product is placed. They can help pick up sounds that indicate a health risk such as cough and/or sneeze. Similarly, temperatures sensor(s) 812 can take temperature readings of locations or of those that come in contact with a product. For example, temperature readings can be taken of the person that delivers the products to an end-user or of the shipping container for a perishable product.

In some embodiments, health certification module 800 can include several other devices, sensors, or combinations of devices and sensors working together to detect viruses. For instance, an ultraviolet (“UV”) lamp and camera could be used to detect fluorescent organisms. A camera could detect discoloration (i.e., indicating fluorescent organisms) in the nearby area while the UV light lights up the nearby area. In some embodiments, the locations (i.e., facilities or vehicles) that a product travels through or within can use aerosol traps to collect air samples. The aerosol traps can then be tested for viruses including COVID-19, chickenpox, and/or tuberculosis. If there is evidence of a virus, the health certification can be updated, as discussed herein.

Self-reporting module(s) 814 can request participants in the life cycle to self-report any health risks. The platform can prompt a participant to periodically report any potential risks. The potential risk may be regarding the participant's health status (i.e., feeling a fever, nauseous, or headache) and/or other potential threat they witness such as a co-worker coughing near a product.

Telehealth module(s) 816 can access health details from various health sources. Telehealth module(s) 816 can connect to telehealth smart applications to determine whether risks exist.

Digital signal processor(s) 820 (DSP) is a microprocessor that executes algorithms to filter, compress, or otherwise process analog signals. For example, the DSP 820 may receive signals from optical sensor(s) 806. DSP 820 may then process the signals and output the signal to encoder(s) 822 for encoding. Encoder(s) 822 can be used to convert digital video data from one format to another.

Notification module(s) 824 may be configured to alert an end-user or participants regarding the health status of a product. Moreover, notification module(s) 824 may perform other tasks such as providing recommendations to keep a safe distance away from the product and/or alerting a participant to self-report health details. Notification module(s) 824 may also communicate with other components of health certification module 800 to determine when to notify of potential health risks.

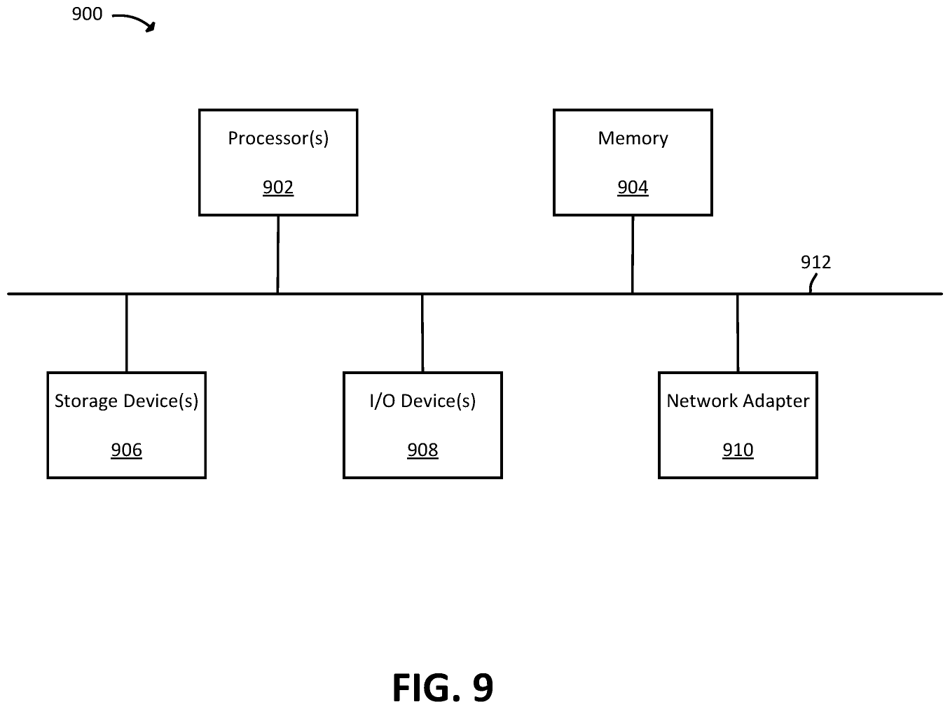

FIG. 9 illustrates an example of a computer system with which embodiments of the present invention may be utilized. The computing system 900 may be a modular reporting device, a reporting system, a server computer, a client computer, a personal computer (PC), a user device, a tablet PC, a laptop computer, a personal digital assistant (PDA), a cellular telephone, an IPHONE, an IPAD, a BLACKBERRY, a processor, a telephone, a web appliance, a network router, switch or bridge, a console, a handheld console, a (handheld) gaming device, a music player, any portable, mobile, handheld device, wearable device, or any machine capable of executing a set of instructions, sequential or otherwise, that specify actions to be taken by that machine.

The computing system 900 may include one or more central processing units (“processors”) 902, memory 904, input/output devices 906 (e.g., keyboard and pointing devices, touch devices, display devices), storage devices 908 (e.g., disk drives), and network adapters 910 (e.g., network interfaces) that are each connected to an interconnect 912. The interconnect 912 is illustrated as an abstraction that represents any one or more separate physical buses, point to point connections, or both connected by appropriate bridges, adapters, or controllers. The interconnect 912, therefore, may include, for example, a system bus, a peripheral component interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1394 bus (i.e., FIREWIRE).

The memory 904 and storage devices 908 are computer-readable storage media that may store instructions that implement at least portions of the various embodiments. In addition, the data structures and message structures may be stored or transmitted via a data transmission medium (e.g., a signal on a communications link). Various communications links may be used (e.g., the Internet, a local area network, a wide area network, or a point-to-point dial-up connection). Thus, computer readable media can include computer readable storage media (e.g. non-transitory media) and computer readable transmission media.

The instructions stored in memory 904 can be implemented as software and/or firmware to program the processor 902 to carry out actions described above. In some embodiments, such software or firmware may be initially provided to the computing system 900 by downloading it from a remote system through the computing system 900 (e.g., via network adapter 910).

The various embodiments introduced herein can be implemented by, for example, programmable circuitry (e.g. one or more microprocessors, programmed with software and/or firmware), or entirely in special-purpose hardwired circuitry (i.e., non-programmable circuitry), or in a combination of such forms. Special-purpose hardwired circuitry may be in the form of, for example, one or more application-specific integrated circuits (ASICs), programmable logic devices (PLDs), field-programmable gate array (FPGAs), etc.

In view of the description herein, there are several embodiments that may developed. For example, a computer-implemented method for determining whether a product was exposed to pathogen particles during production and distribution stages, the method comprising: acquiring pathogen related data from sensors, wherein the sensors are operable to monitor an environment around the product, and wherein the sensors are located at a first location associated with a stage of a plurality of stages; determining, based on the pathogen related data from the sensors, a probability that pathogen particles contacted the product; in response to determining that the probability that pathogen particles did contact the product is above a pre-determined threshold probability value, associating, an unsafe pathogen certificate with the stage, the unsafe pathogen certificate being an indicator that the product should not contact another object or person; and appending a recommendation report to a web-based application, based on the unsafe pathogen certificate, the recommendation report being related to limiting the spread of the pathogen particles.

Further to the example above, wherein the sensors are operable to detect H1N1 pathogens and wherein the sensors include a conductive polymer placed between electrodes, the method further comprising: detecting, by the electrodes, a change in voltage caused by H1N1 pathogens adhering to the conductive polymer. The conductive polymer is operable to adhere to any of nitric oxide, acetone, ammonia, or any combination thereof. The sensors are any of optical sensors, potentiometric sensors, impediametric sensors, amperometric sensors, piezoelectric sensors, magnetic sensors, thermometric sensors, or any combination thereof. The example including appending the recommendation report to a blockchain ledger. Determining the probability that pathogen particles contacted the product further comprises: parsing the pathogen related data through a pipeline of deep learning models, wherein an output from a first deep learning model becomes an input to a second deep learning model, and wherein the pipeline includes any two of a model that uses a frequency of occurrence of a term in a document, a model that derives topic features, a clustering based model, an iterative clustering based model, or a model that uses tree-structures for attribute classification. The pathogen particles includes pathogens caused by a common cold, flu, meningitis, herpes, chicken pox, measles, norovirus, rotavirus, hepatis, yellow fever, dengue fever, HIV, AIDS, COVID-19, or any combination thereof. The first example including acquiring GPS data representative of the first location. Acquiring pathogen related data from sensors further comprises: acquiring data from sensors onboard wearable devices, wherein the wearable devices are associated with users that contact the product, and wherein the sensors onboard wearable devices can be any of heart rate sensors, step trackers, oximeters, or any combination thereof. Associating the unsafe pathogen certificate with the product further comprises: associating the product with a quick response (QR) code, wherein the QR code grants access to the web-based application and the recommendation report. The recommendation report includes the data acquired from the sensors and the pathogen certificate associated with the stage. The stage is a first stage of the plurality of stages, the method further comprising: acquiring pathogen related data from sensors, wherein the sensors are operable to monitor an environment around the product, and wherein the sensors are located at a second location associated with a second stage of the plurality of stages; determining, based on the pathogen related data from the sensors located at the second location, the probability that pathogen particles contacted the product at the first location and not the second location; associating, based on the determination that the probability that pathogen particles did contact the product at the first location is higher than the pre-determined threshold probability and not higher than the pre-determined threshold value at the second location, the an unsafe pathogen certificate for the first location and a safe pathogen certificate for the second location, the safe pathogen certificate being an indicator that the product can contact another object or person. Associating the life cycle of the product with an unsafe pathogen certificate, based on associating the first location with an unsafe pathogen certificate. Determining a number of times pathogen particles contacted the product in the first location and the second location; determining that the number of times is above a threshold value; and associating the life cycle of the product with an unsafe pathogen certificate. Determining a number of times pathogen particles contacted the product in the first location and second location; determining that the number of times is below a threshold value; and associating the life cycle of the product with a safe pathogen certificate. The stage is a first stage of the plurality of stages, and wherein associating a pathogen certification with the product further comprises: acquiring pathogen related data from sensors, wherein the sensors are located at a second location associated with a second stage of the plurality of stages; determining, based on the pathogen related data from the sensors located at the second location, the probability that pathogen particles did contact the product at the first location and the second location is below the pre-determined threshold; associating, based on the determination that the probability that pathogen particles did contact the product at the first location and the second location is below the pre-determined threshold, a safe pathogen certificate for the first location and a safe pathogen certificate for the second location. Associating, the life cycle of the product with a safe pathogen certificate, based on associating the first location and the second location with a safe pathogen certificate. Providing recommendations further comprises: analyzing the number of days that have passed since the pathogen particles have come in to contact with the product.

In another example, a computer-implemented method of providing a health certificate for a product, the method comprising: integrating, by a host application at a computing device associated with a first user, a plug-in, the plug-in capable of providing instructions to the computing device, the instructions including: acquiring, at the computing device associated with the first user, data from a first set of sensors, the first set of sensors being operable to monitor an environment around the product and located at a first location of a life cycle of the product, the data being related to health risks; analyzing the data from the first set of sensors; identifying health risks, based on the analysis of the data from the first set of sensors; and establishing the health certificate for the first location, based on the identified health risks; and displaying, by the computing device, the health certificate. The health risks are associated with pathogens caused by a common cold, flu, meningitis, herpes, chicken pox, measles, norovirus, rotavirus, hepatis, yellow fever, dengue fever, HIV, AIDS, COVID-19, or any combination thereof. Identifying health risks further comprises: determining there are no health risks associated with the data from the first set of sensors; and establishing a safe health certificate for the first location. Identifying health risks further comprises: determining there are health risks associated with the data from the first set of sensors; and establishing an unsafe health certificate for the first location. Identifying a plurality of health risks; assigning a score for each identified health risk of the plurality of health risks; summing the scores provided to each health risk of the plurality of health risks; determining that the sum of the scores is higher than a threshold value; and establishing an unsafe health certificate for the first location. Identifying a plurality of health risks; assigning a score for each identified health risk of the plurality of health risks; summing the scores provided to each health risk of the plurality of health risks; determining that the sum of the scores is lower than a threshold value; and establishing a safe health certificate for the first location.

In another example, a health certification system comprising: a plurality of sensors operable to monitor an environment around a product, acquire pathogen related data from the environment around the product, and transmit the pathogen related data to a server, wherein the plurality of sensors is located at locations associated with a plurality of stages; the server communicatively coupled to the plurality of sensors, the server comprising: a processor; and a memory having instructions stored thereon that, when executed by the processor, cause the processor to: receive the pathogen related data from the plurality of sensors; determine, based on the pathogen related data from the sensors, a probability that pathogen particles contacted the product; associate, based on the probability that pathogen particles contacted the product, a pathogen certificate with the stage; and provide recommendations based on the pathogen certificate, the recommendations being related to limiting the spread of pathogen and an application operable to be executed on a user device and display the pathogen certificate and recommendations on a page of the application. The plurality of sensors are any of thermal sensors, optical sensors, air pollution sensors, chemical sensors, biosensors, optical sensors, potentiometric sensors, impediametric sensors, amperometric sensors, piezoelectric sensors, magnetic sensors, and thermometric sensors. or any combination thereof. The plurality of sensors comprise gold nanoislands, wherein the refractive index of the gold nanoislands change when in the presence of pathogen particles.

In another example, a computer-implemented method for determining whether a product was exposed to pathogen particles during a packaging stage, the method comprising: acquiring pathogen related data from sensors, wherein the sensors are operable to monitor each layer of packaging among a plurality of packaging layers that are applied to the product; determining, based on the pathogen related data from the sensors, the probability that pathogen particles contacted a packaging layer among the plurality of packaging layers; in response to determining that the probability that pathogen particles did contact the packaging layer is above a pre-determined threshold, associating an unsafe pathogen certificate with the product, the unsafe pathogen certificate being an indicator that the product should not contact another object or person; and providing recommendations based on the unsafe pathogen certificate the recommendations being related to limit the spread of pathogen. The plurality of layers is applied sequentially. The sensors monitor each layer of the plurality of packaging layers prior to application on the product.

From the foregoing, it will be appreciated that specific embodiments of the invention have been described herein for purposes of illustration, but that various modifications may be made without deviating from the scope of the invention. Accordingly, the invention is not limited except as by the appended claims.

1. A computer-implemented method for determining whether a product was exposed to pathogen particles during production and distribution stages, the method comprising:
acquiring pathogen related data from sensors, wherein the sensors are operable to monitor an environment around the product, and wherein the sensors are located at a first location associated with a stage of a plurality of stages;
determining, based on the pathogen related data from the sensors, a probability that pathogen particles contacted the product;
in response to determining that the probability that pathogen particles did contact the product is above a pre-determined threshold probability value, associating, an unsafe pathogen certificate with the stage, the unsafe pathogen certificate being an indicator that the product should not contact another object or person; and
appending a recommendation report to a web-based application, based on the unsafe pathogen certificate, the recommendation report being related to limiting the spread of the pathogen particles.

2. The method of claim 1, wherein the sensors are operable to detect H1N1 pathogens and wherein the sensors include a conductive polymer placed between electrodes, the method further comprising:
detecting, by the electrodes, a change in voltage caused by H1N1 pathogens adhering to the conductive polymer.

3. The method of claim 2, wherein the conductive polymer is operable to adhere to any of nitric oxide, acetone, ammonia, or any combination thereof.

4. The method of claim 1, wherein the sensors are any of optical sensors, potentiometric sensors, impediametric sensors, amperometric sensors, piezoelectric sensors, magnetic sensors, thermometric sensors, or any combination thereof.

5. The method of claim 1 further comprising:
appending the recommendation report to a blockchain ledger.

6. The method of claim 1, wherein determining the probability that pathogen particles contacted the product further comprises:
parsing the pathogen related data through a pipeline of deep learning models, wherein an output from a first deep learning model becomes an input to a second deep learning model, and wherein the pipeline includes any two of a model that uses a frequency of occurrence of a term in a document, a model that derives topic features, a clustering based model, an iterative clustering based model, or a model that uses tree-structures for attribute classification.

7. The method of claim 1, wherein the pathogen particles includes pathogens caused by a common cold, flu, meningitis, herpes, chicken pox, measles, norovirus, rotavirus, hepatis, yellow fever, dengue fever, HIV, AIDS, COVID-19, or any combination thereof.

8. The method of claim 1, further comprising:
acquiring GPS data representative of the first location.

9. The method of claim 1, wherein acquiring pathogen related data from sensors further comprises:
acquiring data from sensors onboard wearable devices, wherein the wearable devices are associated with users that contact the product, and wherein the sensors onboard wearable devices can be any of heart rate sensors, step trackers, oximeters, or any combination thereof.

10. The method of claim 1, wherein associating the unsafe pathogen certificate with the product further comprises:
associating the product with a quick response (QR) code, wherein the QR code grants access to the web-based application and the recommendation report.

11. The method of claim 1, wherein the recommendation report includes the data acquired from the sensors and the pathogen certificate associated with the stage.

12. The method of claim 1, wherein the stage is a first stage of the plurality of stages, the method further comprising:
acquiring pathogen related data from sensors, wherein the sensors are operable to monitor an environment around the product, and wherein the sensors are located at a second location associated with a second stage of the plurality of stages;
determining, based on the pathogen related data from the sensors located at the second location, the probability that pathogen particles contacted the product at the first location and not the second location;
associating, based on the determination that the probability that pathogen particles did contact the product at the first location is higher than the pre-determined threshold probability and not higher than the pre-determined threshold value at the second location, the an unsafe pathogen certificate for the first location and a safe pathogen certificate for the second location, the safe pathogen certificate being an indicator that the product can contact another object or person.

13. The method of claim 12 further comprising:
associating the life cycle of the product with an unsafe pathogen certificate, based on associating the first location with an unsafe pathogen certificate.

14. The method of claim 12 further comprising:
determining a number of times pathogen particles contacted the product in the first location and the second location;
determining that the number of times is above a threshold value; and
associating the life cycle of the product with an unsafe pathogen certificate.

15. The method of claim 12 further comprising:
determining a number of times pathogen particles contacted the product in the first location and second location;
determining that the number of times is below a threshold value; and
associating the life cycle of the product with a safe pathogen certificate.

16. The method of claim 1, wherein the stage is a first stage of the plurality of stages, and wherein associating a pathogen certification with the product further comprises:
acquiring pathogen related data from sensors, wherein the sensors are located at a second location associated with a second stage of the plurality of stages;
determining, based on the pathogen related data from the sensors located at the second location, the probability that pathogen particles did contact the product at the first location and the second location is below the pre-determined threshold;
associating, based on the determination that the probability that pathogen particles did contact the product at the first location and the second location is below the pre-determined threshold, a safe pathogen certificate for the first location and a safe pathogen certificate for the second location.

17. The method of claim 16 further comprising:
associating, the life cycle of the product with a safe pathogen certificate, based on associating the first location and the second location with a safe pathogen certificate.

18. The method of claim 1, wherein providing recommendations further comprises:
analyzing the number of days that have passed since the pathogen particles have come in to contact with the product.

19. A computer-implemented method of providing a health certificate for a product, the method comprising:
integrating, by a host application at a computing device associated with a first user, a plug-in, the plug-in capable of providing instructions to the computing device, the instructions including:
acquiring, at the computing device associated with the first user, data from a first set of sensors, the first set of sensors being operable to monitor an environment around the product and located at a first location of a life cycle of the product, the data being related to health risks;
analyzing the data from the first set of sensors;
identifying health risks, based on the analysis of the data from the first set of sensors; and
establishing the health certificate for the first location, based on the identified health risks;
displaying, by the plug-in on a display of the computing device, the health certificate; and
appending a recommendation report to the plug-in, based on the health certificate, the recommendation report being related to limiting the spread of the health risks.

20. The method of claim 19, wherein the health risks are associated with pathogens caused by a common cold, flu, meningitis, herpes, chicken pox, measles, norovirus, rotavirus, hepatis, yellow fever, dengue fever, HIV, AIDS, COVID-19, or any combination thereof.

21. The method of claim 19, wherein identifying health risks further comprises:
determining there are no health risks associated with the data from the first set of sensors; and
establishing a safe health certificate for the first location.

22. The method of claim 19, wherein identifying health risks further comprises:
determining there are health risks associated with the data from the first set of sensors; and
establishing an unsafe health certificate for the first location.

23. The method of claim 19 further comprising:
identifying a plurality of health risks;
assigning a score for each identified health risk of the plurality of health risks;
summing the scores provided to each health risk of the plurality of health risks;
determining that the sum of the scores is higher than a threshold value; and
establishing an unsafe health certificate for the first location.

24. The method of claim 19, wherein identifying health risks further comprises:
identifying a plurality of health risks;
assigning a score for each identified health risk of the plurality of health risks;
summing the scores provided to each health risk of the plurality of health risks;
determining that the sum of the scores is lower than a threshold value; and
establishing a safe health certificate for the first location.

25. A health certification system comprising:
a plurality of sensors operable to monitor an environment around a product, acquire pathogen related data from the environment around the product, and transmit the pathogen related data to a server, wherein the plurality of sensors is located at locations associated with a plurality of stages;
the server communicatively coupled to the plurality of sensors, the server comprising:
a processor; and
a memory having instructions stored thereon that, when executed by the processor, cause the processor to:
receive the pathogen related data from the plurality of sensors;
determine, based on the pathogen related data from the sensors, a probability that pathogen particles contacted the product;
associate, based on the probability that pathogen particles contacted the product, a pathogen certificate with the stage; and
provide recommendations based on the pathogen certificate, the recommendations being related to limiting the spread of pathogen and
a web-based application operable to be executed on a user device and display the pathogen certificate and recommendations on a page of the application.

26. The electronic device of claim 25, wherein the plurality of sensors are any of thermal sensors, optical sensors, air pollution sensors, chemical sensors, biosensors, optical sensors, potentiometric sensors, impediametric sensors, amperometric sensors, piezoelectric sensors, magnetic sensors, and thermometric sensors. or any combination thereof.

27. The electronic device of claim 25, wherein the plurality of sensors comprise gold nanoislands, wherein the refractive index of the gold nanoislands change when in the presence of pathogen particles.

28. A computer-implemented method for determining whether a product was exposed to pathogen particles during a packaging stage, the method comprising:
acquiring pathogen related data from sensors, wherein the sensors are operable to monitor each layer of packaging among a plurality of packaging layers that are applied to the product;
determining, based on the pathogen related data from the sensors, the probability that pathogen particles contacted a packaging layer among the plurality of packaging layers;
in response to determining that the probability that pathogen particles did contact the packaging layer is above a pre-determined threshold, associating an unsafe pathogen certificate with the product, the unsafe pathogen certificate being an indicator that the product should not contact another object or person; and
appending a recommendation report to a web-based application, based on the unsafe pathogen certificate, the recommendation report being related to limiting the spread of pathogen.

29. The method of claim 28, wherein the plurality of layers is applied sequentially.

30. The method of claim 28, wherein the sensors monitor each layer of the plurality of packaging layers prior to application on the product.
